(19) United States
(12) Patent Application Publication
(10) Pub. No.: US 2026/0089443 A1
(43) Pub. Date: Mar. 26, 2026

(54) In-Ear, Over-Ear Headphones or Speaker with Real-Time Audio Multi-Frequency Conversion (Wired or Wireless)

(71) Applicant: Laura Gagliardi, Los Angeles, CA (US)

(72) Inventor: Laura Gagliardi, Los Angeles, CA (US)

(21) Appl. No.: 19/277,262

(22) Filed: Jul. 22, 2025

Related U.S. Application Data
(60) Provisional application No. 63/699,723, filed on Sep. 26, 2024.

(51) Int. Cl. H04R 3/04; A61M 21/02; G06F 3/16; A61M 21/00

(52) U.S. Cl. CPC H04R 3/04; A61M 21/02; G06F 3/165; A61M 2021/0022; A61M 2021/0027; A61M 2205/8206

(57) ABSTRACT

This invention relates to wireless or wired in-ear, over-ear headphones or speaker systems integrated with a digital signal processor (DSP) capable of real-time audio frequency conversion. The device enables users to automatically retune standard audio signals—typically at 440 Hz—to alternative frequencies such as 432 Hz, 528 Hz, 40 Hz, and others with documented therapeutic benefits. The system supports Bluetooth and wired input and includes both physical controls and a companion mobile application for frequency selection, sound customization, firmware updates, and battery monitoring. A tactile vibrational feedback feature extends accessibility to users with auditory impairments. The invention offers a portable, hardware-based solution for sound therapy without requiring audio file conversion or software post-processing.

### FIELD OF THE INVENTION

This invention pertains to the field of wireless or wired audio devices, specifically focusing on in-ear / over-ear headphones or speakers equipped with a digital signal processor (DSP) for real-time audio frequency conversion. The invention provides users with the ability to experience the therapeutic benefits of specific frequencies (e.g., 432 Hz, 528 Hz, and others) through music, enhancing both mental and physical well-being.

### BACKGROUND OF THE INVENTION

Modern music is typically tuned to 440 Hz, but research suggests that other frequencies, such as 432 Hz, may offer greater therapeutic benefits.

Current methods for converting music to alternate frequencies require manual processing through software, making it inaccessible for general users.

This invention provides an effortless hardware-based solution that automatically converts audio frequencies in real-time, allowing users to experience the benefits of sound therapy through a portable, user-friendly device.

### SUMMARY OF THE INVENTION

The invention introduces wireless or wired in-ear/over-ear headphones or speakers equipped with a DSP capable of converting audio frequencies in real-time from 440 Hz to multiple healing frequencies (e.g., 432 Hz, 528 Hz, 396 Hz).

The headphones or speakers are compatible with any audio source and utilize either Bluetooth technology for seamless connectivity or wired connection via available audio input for several devices.

The system supports both physical controls and a companion mobile app, providing users with customizable sound profiles, frequency selection, and firmware updates.

The real-time conversion enables users to integrate sound therapy into their daily lives without altering the original music files.

### BRIEF DESCRIPTION OF THE DRAWINGS

FIG. 1 is an illustration of the wireless headphones according to one embodiment, shown from a front and a side perspective view with close up of the switch and of the over-the-ear piece.

FIG. 2 is a schematic diagram of the signal processing pathway next to a stylized illustration of the headphones.

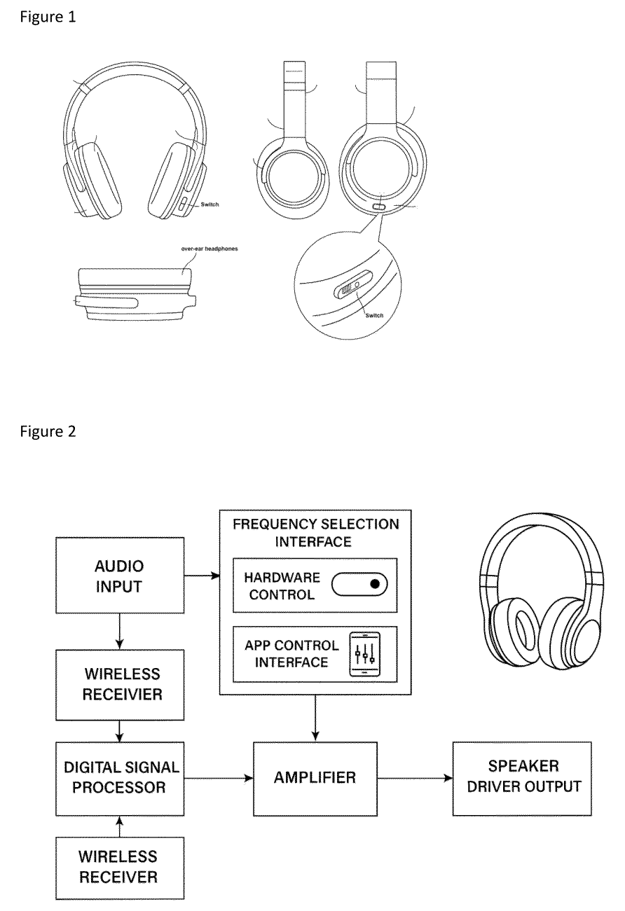

### DETAILED DESCRIPTION

### Device Overview

The wireless / wired in-ear / over-ear headphones or speakers include a digital signal processor (DSP) that automatically detects and converts incoming audio frequencies in real-time.

The DSP is programmed to convert standard 440 Hz music to a variety of healing frequencies, such as 432 Hz for relaxation and 528 Hz for emotional balance.

The conversion is applied to audio streams from any connected device (smartphones, tablets, etc.) without the need for additional software.

### Wireless Technology

The device utilizes Bluetooth 5.0/6.0 for seamless connectivity to external devices with a range of up to 10 meters, or a wired connection via audio input.

The DSP processes the audio signal before it reaches the user, ensuring frequency conversion is applied in real-time without audio quality degradation.

### Audio Frequency Conversion

The key feature of the invention is the real-time conversion of audio frequencies. The DSP adjusts the incoming audio frequency based on user selection, allowing them to switch between frequencies such as: 432 Hz: Promotes relaxation and emotional healing. 528 Hz: Commonly referred to as the "Love Frequency," associated with cellular repair and transformation. 40 Hz: Linked to cognitive function and memory enhancement.

The conversion is seamless, ensuring the user experiences the full therapeutic benefits of these frequencies without distorting the original audio.

### User Interface and Control

The headphones/speakers offer two control systems: Physical Controls: Users can toggle between different frequency modes using physical buttons located on the headphones. These buttons allow for easy switching between modes such as 432 Hz, 528 Hz, 40 Hz and others. Mobile App Interface: The companion app, available on iOS and Android, offers more advanced controls. Users can customize sound profiles, adjust frequency settings, and perform firmware updates via the app. The app also includes features like battery monitoring and vibrational feedback for users with hearing impairments.

### Battery & Power Management

The headphones are powered by a 500mAh rechargeable battery that supports up to 10 hours of playback.

The device includes a power-saving mode that activates after a period of inactivity to extend battery life.

Charging is supported through both USB-C and wireless charging options, if needed.

### Vibrational Feedback

For users who are deaf or hard of hearing, the headphones include a vibrational feedback function. This allows users to feel the sound frequencies through tactile feedback, providing a sensory experience similar to vibroacoustic therapy.

This feature makes the device accessible to a wider audience, including individuals with auditory impairments.

### Applications

The device can be used in various therapeutic settings, such as: Cognitive Enhancement: 40 Hz frequency stimulation for improving memory and cognitive function. Stress Reduction: 432 Hz for relaxation and stress relief. Emotional Healing: 528 Hz for promoting emotional balance and well-being.

1. A wireless or wired in-ear or over-ear headphone or speaker system comprising:
a digital signal processor (DSP) configured to convert incoming audio frequencies from a standard 440 Hz tuning to 432 Hz, 528 Hz, 40 Hz, and other selectable frequencies in real-time; wireless connectivity via Bluetooth or a wired connection for receiving audio signals from a source device; physical buttons and a mobile application for selecting frequencies, customizing audio settings, and managing firmware updates; and a rechargeable battery capable of supporting up to 10 hours of playback.

2. The wireless or wired in-ear or over-ear headphones or speakers of claim 1, wherein the DSP adjusts audio frequencies in real-time based on user selection.

3. The wireless or wired in-ear or over-ear headphones or speakers of claim 1, further comprising a vibrational feedback system for users with auditory impairments.

4. The wireless or wired in-ear or over-ear headphones or speakers of claim 1, wherein the mobile application allows users to customize sound profiles, manage firmware updates, and monitor battery life.
